(19) United States
(12) Patent Application Publication
(10) Pub. No.: US 2007/0167826 A1
(43) Pub. Date: Jul. 19, 2007

(54) Apparatuses for thermal management of actuated probes, such as catheter distal ends

(76) Inventors: Warren Lee, Niskayuna, NY (US); Douglas Glenn Wildes, Ballston Lake, NY (US); Weston Blaine Griffin, Guilderland, NY (US); Mirsaid Seyed-Bolorforosh, Guilderland, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/330,378

(22) Filed: Jan. 11, 2006

Related U.S. Application Data
(63) Continuation-in-part of application No. 11/289,926, filed on Nov. 30, 2005.

(51) Int. Cl. A61B 8/14

(52) U.S. Cl. 600/463

(57) ABSTRACT

Alternative embodiments are provided, for use individually or in combination, for thermal management of catheter distal ends and other types of probes such as may be used in catheter and other instruments' diagnostic and interventional devices. In an exemplary embodiment, a catheter distal end (1000) comprises an ultrasound imaging assembly (1003) that comprises an actuator (1004), a drive shaft (1006), a section (1008) of an interconnect (1010), and a transducer assembly (1009). A circulation fin (1018) optionally may be affixed to the bottom (1016) of transducer assembly (1009), and extends into a defined space (1017) to enhance circulation of acoustic transmission medium that is in the defined space (1017). This disperses heat from the actuator (1004) and the transducer assembly (1009). A similar fin (1018) may also, or alternatively, be positioned on the section (1008) of interconnect (1010). Other thermal management approaches also are disclosed.

### CROSS-REFERENCE TO RELATED APPLICATIONS

This application is a continuation in part of application Ser. No. 11/289,926, filed Nov. 30, 2005. This application is also related to concurrently filed application Ser. No. ______, filed Jan. 11, 2006, and entitled Method of Manufacture of Catheter Tips, Including Mechanically Scanning Ultrasound Probe Catheter Tip, And Apparatus Made By The Method, and this application is also related to concurrently filed application Ser. No. ______, filed Jan. 11, 2006, and entitled Apparatus for Catheter Tips, Including Mechanically Scanning Ultrasound Probe Catheter Tip.

### FIELD OF THE INVENTION

The field of the invention is diagnostic and interventional probes, including catheters, and more particularly thermal management of ultrasonic probes for a catheter system.

### BACKGROUND OF THE INVENTION

Ultrasound imaging of living human beings and animals has advanced in recent years in part due to advances in technologies related to computer data storage, transfer and analysis. Other advances, in the fields of component miniaturization and transducer design and composition, likewise have contributed to the advances in ultrasound imaging devices and methods.

Such advances have provided a foundation for development of various approaches to real time three-dimensional (“RT3D”) ultrasonic imaging, including those that use a catheter-based ultrasound probe. Real time three-dimensional ultrasonic imaging from a unit housed in a catheter offers many advantages for conducting exacting diagnostic and interventional procedures. Accordingly, improvements in this field are expected to offer substantial cost effectiveness and other benefits for medical diagnostics and interventions.

More generally, probes, such as catheter distal ends, that comprise diagnostic and/or interventional devices may be relatively small in overall volume and yet may comprise heat-generating components. Unless there is effective thermal management, these probes may have external areas that reach an unacceptable temperature when used within a human or animal body.

Therefore, there is a need to consider how to manage heat developed by various components of a probe, such as a catheter distal end. For example, a heat-generating actuator may be provided within a catheter tip, such as for movement of a transducer or other component. FIG. 1 depicts the surface temperature of a 3-millimeter diameter SMOOVY® motor during operation under a representative load. Such motor is of a size that it may be utilized in catheter distal ends to power movement of a transducer array for ultrasonic imaging. After approximately 120 seconds of operation, the surface temperature rises steeply from room temperature to about 70 degrees Celsius. It maintains this temperature during its operation (to about 400 seconds, at which time power is disconnected), and then surface temperature drops as shown in FIG. 1.

If such micromotor were installed in a catheter distal end to power movement of a transducer array, for example as part of an ultrasonic imaging catheter tip, the heat generated by its operation would need to be dissipated without creating an unacceptably hot area on the surface of the catheter tip. Particularly, the International Electrotechnical Commission (IEC) has established maximum temperature limits that may not be exceeded by devices, such as catheters, that are inserted into a human body. Thus, a need exists in the art to develop apparatuses and methods for appropriate heat dissipation of heat developed in probes, such as in catheter distal ends, for example an ultrasonic imaging catheter tip that utilizes a micromotor-type actuator for powering movement of a transducer array.

### BRIEF DESCRIPTION OF THE DRAWINGS

Features, aspects and advantages of embodiments of the present invention will become better understood when the following detailed description is read with reference to the accompanying drawings in which like characters represent like parts, wherein:

FIG. 1 provides a graph that depicts the surface temperature of a miniature motor during operation under a representative load.

FIG. 2 is a side view with cut-away, and partially schematic representation of a catheter distal end that is integral or attached to a catheter body, and connected to a catheter control system.

FIG. 3A is a side view with cut-away that provides an enlarged view of components within the dashed area of FIG. 2, however illustrating an alternative embodiment. FIG. 3B, presents a schematic partial cut-away side view of a catheter distal end that depicts aspects of thermal management embodiments.

FIG. 4 is a side view with cut-away that illustrates an alternative embodiment in which a thermally conductive metal layer is one component of a catheter outer wall that surrounds a heat-generating actuator.

FIG. 5 illustrates an alternative embodiment comprising a heat-generating actuator comprising five equally spaced apart mounts that contact an inner surface of a catheter or catheter tip outer wall.

FIG. 6 illustrates an alternative embodiment in which a thermally conductive fluid surrounds a heat-generating actuator.

FIG. 7 illustrates an alternative embodiment in which a thermally conductive fluid surrounds a heat-generating actuator, and a thermally conductive metal layer is provided in an outer wall.

FIG. 8 illustrates an alternative embodiment in which a thermally conductive fluid surrounds a heat-generating actuator, and a thermally conductive metal braid is provided in an outer wall.

FIG. 9 illustrates an alternative embodiment in which a thermally conductive fluid surrounds a heat-generating actuator, and a propeller is provided on a drive shaft of an actuator.

FIG. 10A and FIG. 10B provide side and bottom views of a catheter distal end comprising an ultrasound imaging assembly in which fins are provided on components to assist in fluid flow for thermal management.

FIG. 11 provides a side and internal view of a catheter distal end as part of a catheter system, in which an open flow is provided for thermal management.

FIG. 12 provides a side and internal view of a catheter distal end as part of a catheter system, in which an closed loop flow is provided for thermal management.

FIG. 13 depicts a catheter distal end, in association with a thermal management control system, that comprises a thermistor or other temperature sensor in association with an actuator.

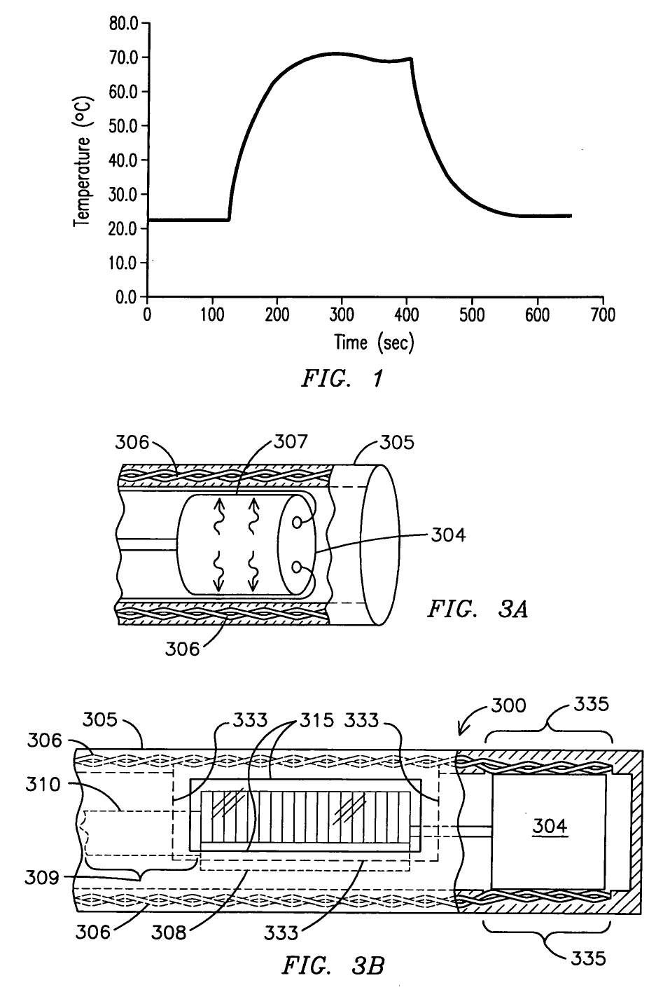

### DETAILED DESCRIPTION OF EMBODIMENTS OF THE INVENTION

Embodiments of the invention provide a number of approaches to solve the problem of achieving effective thermal management of probes, such as catheter distal ends, that comprise heat-generating components. Further, these approaches may be combined in certain embodiments to achieve a desired result. Specific disclosed examples, not meant to be limiting, relate to ultrasound imaging functionality in a catheter distal end that comprises a transducer and an actuator, where both such components generate heat during operation. However, notwithstanding the examples and disclosures herein, it is understood that various aspects for thermal management may be applied for cooling any of a variety of component arrangements in a probe such as a catheter distal end.

By “catheter distal end” is meant a terminus section of a catheter inserted into a human or animal that comprises assembled components to conduct diagnostic and/or interventional procedures. Examples of such procedures include catheters having imaging functionalities (e.g., ultrasound imaging) and/or having ablation and recanalization functionalities (e.g., balloon angioplasty, laser ablation angioplasty, balloon embolectomy, aspiration embolectomy, thermal or RF ablation, abrasion, and drilling). Depending on the design and method of fabrication, a catheter distal end may comprise: a distal region of a unitary catheter structure that holds those assembled components; a catheter tip as that term is defined herein; and a hybrid structure in which an assemblage comprising less than all of the components comprising the diagnostic and/or interventional device is attachable to the remainder of the catheter body.

In the present application, by “catheter tip” is meant a structure comprising components that may provide one or more diagnostic and/or interventional functionalities, where that structure is attachable to a catheter body (the particular catheter body lacking such functionality, and adapted to receive the catheter tip to form a functional catheter). Further, a “catheter tip assembly” may comprise a particular catheter tip, and additionally comprise a length of an interconnect adapted to pass through such a catheter body to connect to a catheter control system to achieve operational connectivity.

In the present disclosure, embodiments of devices are provided that are suitable for intracardiac echocardiography (ICE). However, this is not meant to be limiting, and the embodiments of the invention apply similarly to non-imaging ultrasound, e.g. ultrasound ablation or ultrasound therapy; or non-ultrasound imaging, e.g. optical or electromagnetic; which could generate as much heat as an actuator, and may likewise benefit from thermal management of heat-generating devices in confined spaces. For example, ultrasound imaging devices utilizing approaches described herein may be incorporated for use in various types of probes that may include catheters in general, such as in catheter distal ends as defined above, and in endoscopes, transesophageal probes, and laparoscopic probes that comprise an actuator. The actuator, for example, may be an electromechanical motor, other type of motor, or other type of actuator. Also, while the following figures are disclosed to comprise catheter distal ends, it is appreciated that the approaches may be applied more broadly to such identified probes.

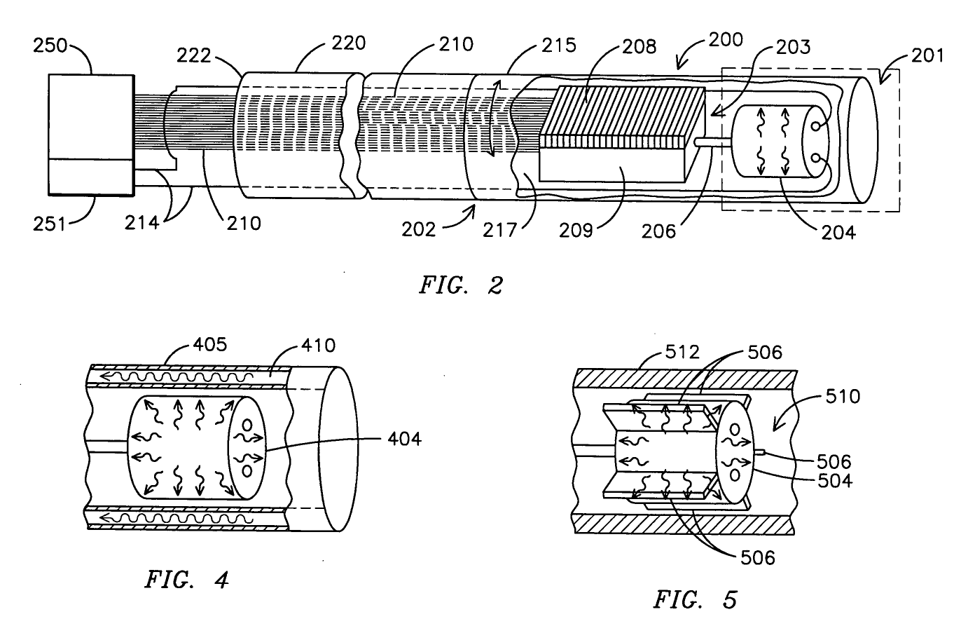

Referring to the figures, FIG. 2 depicts a catheter distal end 200, having a distal end 201 and a proximal end 202, is integral or attached to a catheter body 220 that, at its proximal end 222, generally is connected to a catheter control system 250. The catheter distal end 200 in FIG. 2 may be part of an integral catheter distal end, or may be a catheter tip that may be attached to a catheter body, as the latter are described in a related application Ser. No. ______, filed Jan. 11, 2006, and entitled Apparatus for Catheter Tips, Including Mechanically Scanning Ultrasound Probe Catheter Tip, incorporated by reference for such teachings and for additional descriptions of components therein.

Catheter distal end 200 comprises an ultrasound imaging assembly 203 that is comprised of an actuator 204, a drive shaft 206, a transducer 208 (shown as a ID array, which is not meant to be limiting), and an interconnect 210, which provides electrical communication between the transducer 208 and the catheter control system 250. A catheter distal end 200 that comprises an ultrasound imaging assembly such as 203 may alternatively be termed an “ultrasonic imaging catheter distal end.” While not meant to be limiting, transducer 208 is one component of a transducer assembly 209 (which includes transducer array assemblies), and may comprise a backing element (not shown) and a drive linkage (not shown) for connection to the drive shaft 206. The actuator 204 is in electrical communication with an external rotary motor controller 251 by conduits 214. The external motor controller 251 is depicted as a component of the catheter control system 250.

The actuator 204 is in mechanical driving relationship via the drive shaft 206, to cause movement of the transducer 208. Typically, the actuator 204 moves the transducer 208 in a back and forth pattern along a defined arc to include a desired volume of adjacent tissue to be imaged. This sweeping back and forth may be about a longitudinal axis parallel with the centerline of the catheter distal end. The transducer 208 obtains a number of two-dimensional images during the sweeping cycle and these images may be combined to generate a three-dimensional image. Repeating this sweeping at specified time intervals may provide real time three-dimensional imaging of the tissue, and this may allow for real time visualization of anatomical processes as well as observation of interventional procedures, including procedures effectuated from the same catheter that houses the ultrasound probe.

The ultrasound imaging assembly 203 is enclosed within a catheter outer wall 215, which defines a defined space 217 within itself. In various embodiments, the actuator 204 may be surrounded by a fluid (not shown), which may also surround the transducer 208 and may have desired properties of an acoustic transmission medium. Generally, fluid used to couple acoustic energy from the transducer 208 to a medium of interest external to the catheter outer wall 215 may also be used to conduct thermal energy away from the actuator 204, and this fluid may also surround the actuator 204. In other embodiments, there may be a more direct relationship between the outer surface of the actuator 204 and the catheter wall 215 (including embodiments with no fluid between these components).

Accordingly, considering the relatively small defined volume 217 within the catheter outer wall 215 and the heat generation capacity of an actuator 204 that may be an electromechanical actuator such as described above, during operation the actuator 204, and more generally the defined space 217, are in need of thermal management devices, methods and systems so that the ultrasound imaging assembly 203 may be used within a human or animal body in conformance with the requirements established by the IEC.

FIG. 3A illustrates one alternative embodiment that may be utilized for thermal management. FIG. 3A provides an enlarged view of components within the dashed area of FIG. 2, however illustrating an alternative embodiment. This alternative embodiment may be used for the ultrasound imaging assembly of FIG. 2 as well as for other probe designs. An actuator 304 is positioned in close thermal contact with a metal reinforcement braid 306 within a catheter outer wall 305. During operation, accordingly, heat generated by the actuator 304 conducts or convects from an actuator outer surface 307 to and is conducted away along the length of the catheter outer wall 305 through the metal reinforcement braid 306 that is a component of the catheter outer wall 305. When there is direct contact between actuator outer surface 307 and the catheter outer wall 305, heat conduction may occur. When there is a space between these elements that is filled with a fluid or a gas, then heat convection may occur across this space, after which heat conduction may occur in the catheter outer wall 305. Thus, it is appreciated that in various embodiments thermal management of heat-generating components in a catheter distal end or a catheter tip is achieved by provision of a catheter outer wall that comprises a metal braid, a metal layer, or another type of thermally conductive material as described herein, in association with design and arrangement of components for effective heat conveyance through the catheter outer wall. Alternatively or in combination with this approach, a heat-conducting fluid may be provided to improve such thermal management by improving heat dissipation from the catheter or catheter tip.

In some alternative embodiments, one or more sections of metal reinforcement of the catheter wall may be directly exposed, that is, is not covered by any other material of the catheter outer wall. This optional alternative allows direct contact and heat transfer between a heat-generating element and the metal reinforcement braid Two examples of this are provided in FIG. 3B, which presents a schematic partial cut-away side view of a catheter distal end 300 that comprises a catheter outer wall 305 within which are positioned an actuator 304 connecting to a transducer 308 (shown as an array, which is not meant to be limiting), and a section 309 of interconnect 310, which provides electrical communication between the transducer 308 and a catheter control system (not shown). Metal reinforcement braid 306 is exposed along a first section 333 that is adjacent and forms a border around a window 315 through which acoustic signals may pass from and to transducer 308. No metal exists in the window 315 itself. In operation heat generated by the transducer 308 convects through a gas or liquid to the exposed braid of first section 333, and thereafter such heat is conducted away through the metal reinforcement braid 306. Also depicted is a second exposed section 335 of metal reinforcement of the catheter outer wall 305 which is not covered by any other material of the catheter outer wall 305. This optional alternative allows direct contact and heat transfer between the actuator 304 and the metal reinforcement braid 306. Thus, sections of optional exposed metal braid may be disposed along these or other sections of a catheter outer wall for various thermal conveyance purposes. It is noted that exposure of sections may be achieved by specific manufacturing to achieve this (such as by forming the wall with sections having metal offset interiorly and exposed, or by not providing any material interior to a centrally positioned metal braid at the desired sections), or by post-manufacture removal of material to achieve the metal exposure. Also, in other embodiments an entire length of a catheter, or a catheter tip, outer wall may comprise exposed metal reinforcement along its interior wall.

Other embodiments comprise such thermal conductivity along, instead of metal reinforcement braids, other metal structures in a probe outer wall. These include, but are not limited to, a solid metal layer in a catheter outer wall, wherein the solid metal layer is thermally conductive. FIG. 4 illustrates one such embodiment, where a thermally conductive metal layer 410 is one component of a catheter outer wall 405, and wherein heat (shown by arrows) may move from an actuator 404 into an through the conductive metal layer 410. Exposed sections of a solid metal layer may be provided similarly to the sections of exposed metal braid described in FIG. 3B.

In other embodiments, an electromechanical actuator may be positioned against a catheter outer wall with two or more motor mounts. For example, FIG. 5 depicts an electromechanical actuator 504 comprising five equally spaced apart mounts 506 that contact an inner surface 510 of a catheter (or catheter tip) outer wall 512. The mounts 506 are attached to an outer surface of electromechanical actuator 504, and heat may be transmitted by conduction through the mounts 506 to the catheter outer wall 512. The catheter outer wall 512 may comprise metal braids, solid conductors or other components as described herein for transmission and dispersal of heat. Also, as shown and discussed for the embodiment of FIG. 3B, metal braid or metal layer of catheter outer wall 512 may be exposed between the motor mounts from the catheter outer wall 512 proximate the actuator 504. Also, whether or not such material is so exposed, additional thermally conductive material may be placed between the motor mounts for improving heat transfer, and/or motor mounts may be adapted to convey heat from the actuator to the catheter outer wall 512. Thermally conductive material includes but is not limited to metals and filled polymers. As noted for FIG. 3A above, thermal conveyance may be effectuated by one or more of convection and conduction.

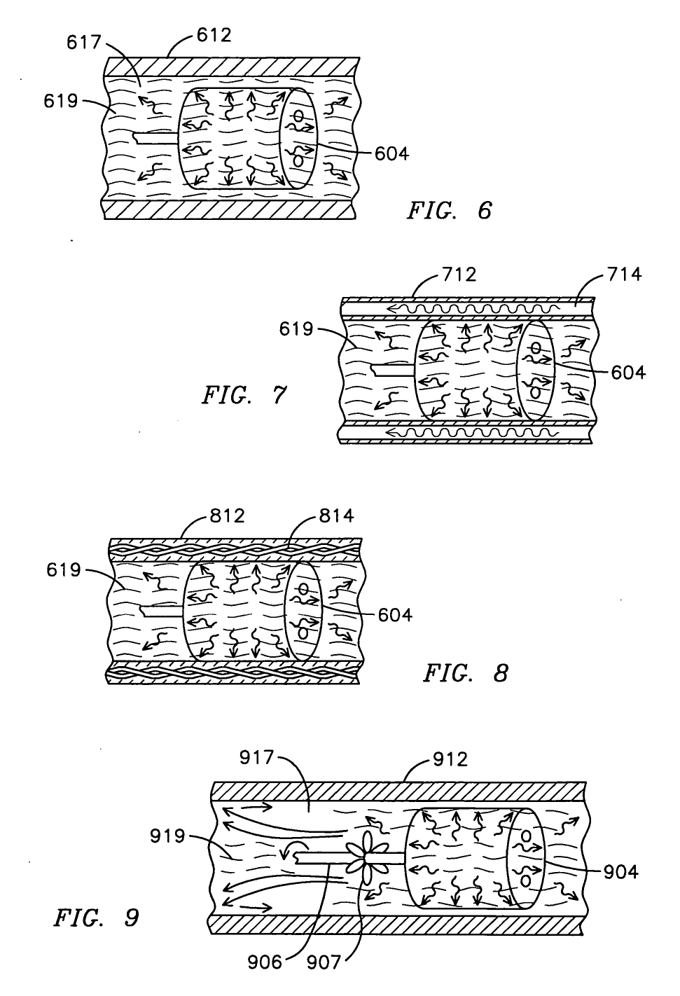

In another embodiment, depicted in FIG. 6, a defined space 617 surrounding an actuator 604 is filled with a thermally conducting, dielectric fluid to provide a dielectric fluid bath, designated as 619. The thermally conducting dielectric fluid bath 619 absorbs and dissipates heat from the actuator. Some of the heat so dissipated may pass to catheter outer wall 612 and be further dissipated along its surface.

More particularly to the latter point, in alternative embodiments depicted in FIGS. 7 and 8, the actuator 604 is immersed in thermally conducting dielectric fluid bath 619 and also is in close thermal contact with respective metal conducting layers in the respective catheter tip catheter outer wall, such as are disclosed above. FIG. 7 depicts a catheter outer wall 712 comprising a thermally conductive metal layer 714, and FIG. 8 depicts a catheter outer wall 812 comprising a thermally conductive metal braid 814. In both examples there is a combined effect of heat dissipation to the thermally conducting dielectric fluid bath 619 and the thermally conductive layer (whether metal layer 714 or metal braid 814) in the respective catheter outer walls 712 and 812. This combined effect of thermal dissipation from the actuator 604 may be appropriate in various embodiments, including catheter tips adapted to more narrow overall size requirements.

The use of a dielectric fluid bath in examples in the above figures and discussion is not meant to be limiting. While a dielectric fluid, such as various perfluorocarbons (examples of which include the 3M® Fluorinert® non-conductive heat transfer fluids), may be utilized, in other embodiments a non-dielectric fluid, such as water and saline, may alternatively be utilized. When using water or saline, which have the advantages of biocompatibility and relatively low viscosity, insulation would be needed for various electrical connections and components. A thermally conductive fluid as may be used in any of the embodiments described herein may or may not be a dielectric fluid, and may optionally be a fluid that transmits acoustic signals within an acceptable range for use in an ultrasonic probe as an acoustic transmission fluid.

As noted above, and as exemplified in FIG. 2, various embodiments of ultrasonic imaging catheter ends may comprise an actuator connected by a drive shaft to a transducer assembly that comprises a transducer or a transducer array. In FIG. 9, a modified drive shaft 906 extending from an actuator 904 comprises a propeller 907 fixedly attached thereto. In such embodiments, the propeller 907 rotates during operation of the actuator 904 and thereby circulates fluid 919 (such as a thermally conducting dielectric fluid) in a defined space 917, providing additional heat dissipation effect for heat generated from the actuator 904. Depending on the position of the propeller 907, this may also act to circulate and accordingly thermally dissipate heat generated by a transducer assembly. Also, the incorporation of a propeller such as propeller 907 may be combined with other approaches, such as providing a metallic conductivity layer (whether braid or solid) in a catheter outer wall 912, and variations of these as disclosed herein.

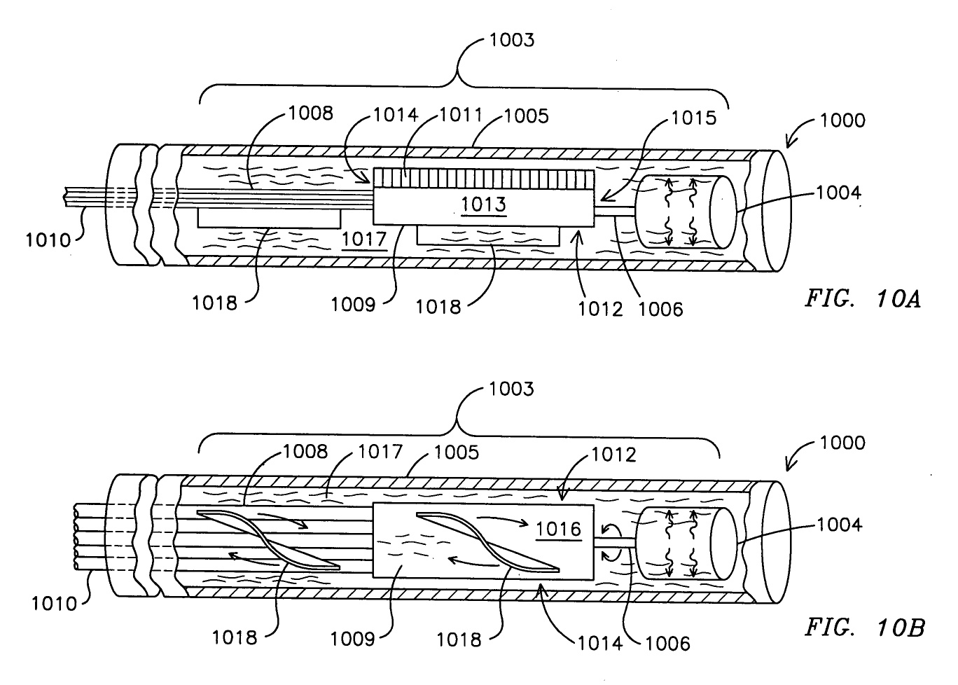

Other embodiments provide thermal management structures on one or more of the ultrasound imaging assembly components described in the embodiment depicted in FIG. 2. For example, FIG. 10A and FIG. 10B provide side and bottom views of a catheter distal end 1000 comprising an ultrasound imaging assembly 1003 that comprises an actuator 1004, a drive shaft 1006, a section 1008 of an interconnect 1010, and a transducer assembly 1009 that comprises a transducer array 1011, a backing element 1013, and a drive linkage 1015 for connection to the drive shaft 1006. The interconnect 1008 provides electrical communication between the transducer array 1011 and a catheter control system (not shown, see FIG. 2). The transducer assembly 1009 comprises opposing sides 1012, 1014 and a bottom 1016 in addition to the side comprising the transducer array 1011. A circulation fin 1018 is affixed to the bottom 1016 of transducer assembly 1009. The circulation fin 1018 extends into a defined space 1017 defined within an outer wall 1005, and during operation enhances circulation of acoustic transmission medium (not shown) that is in the defined space 1017. In various embodiments, the circulation fin 1018 may be designed appropriately to provide a circulation of the acoustic transmission medium within the defined space 1017 around the transducer assembly 1009.

More generally, embodiments may comprise one or more circulation fins such as 1018 on the opposing sides 1012, 1014 and/or the bottom 1016 of the transducer assembly 1009. Also, in various embodiments, a circulation fin such as 1018 additionally may be attached to one or more surfaces of the interconnect 1008 along a portion of the interconnect sufficiently near the transducer array assembly that is subject to rotating motion as the transducer assembly 1009 also rotates during scanning operations. FIGS. 10A and 10B provide one exemplary optional circulation fin 1018 disposed on interconnect 1008. While considered an optional aspect of the embodiment in FIGS. 10A and 10B, it also is appreciated that in some embodiments one or more circulatory fins such as 1018 may be provided on an interconnect whilst no fins are provided on an attached transducer assembly (not shown, but represented in FIGS. 10A and 10B by elimination of the fin 1018 on transducer assembly 1009).

Also, it is appreciated that the components themselves, such as the transducer assembly 1009 and the interconnect 1008 in FIGS. 10A and 10B, may serve to circulate the fluid without requiring fin(s). That is, the shape of the component itself may be, or may be designed, to achieve a desired level of circulation for thermal management.

In another embodiment, depicted in FIG. 11, a catheter distal end 1100 comprises one or more outlets 1111 at or near its physical distal end 1101. Also provided is an inlet 1102 into a defined space 1105 leading from a supply conduit 1103 extending from a supply source (not shown). A seal 1119 across the proximal end of catheter distal end 1100 prevents passage of fluid into the catheter body proximal to the seal 1119. The seal 1119 may be of any type described herein and in related application Ser. No. ______, filed Jan. 11, 2006, and entitled Apparatus for Catheter Tips, Including Mechanically Scanning Ultrasound Probe Catheter Tip, which is incorporated by reference specifically for such teachings. During operation, a suitable fluid (represented by arrows) is flowed from the supply source through the conduit 1103 through the inlet 1102 and into the defined space 1105. There the fluid passes around transducer assembly 1109, around actuator 1104, and exits through the one or more outlets 1111. This provides an open loop flushable catheter system for cooling of the components in the catheter distal end 1100.

In such embodiments in which the fluid may pass into a body space, the fluid is required to be biocompatible. By this is meant that the fluid is approved for intravenous or intracardiac injection. One example of a biocompatible fluid is sterile saline.

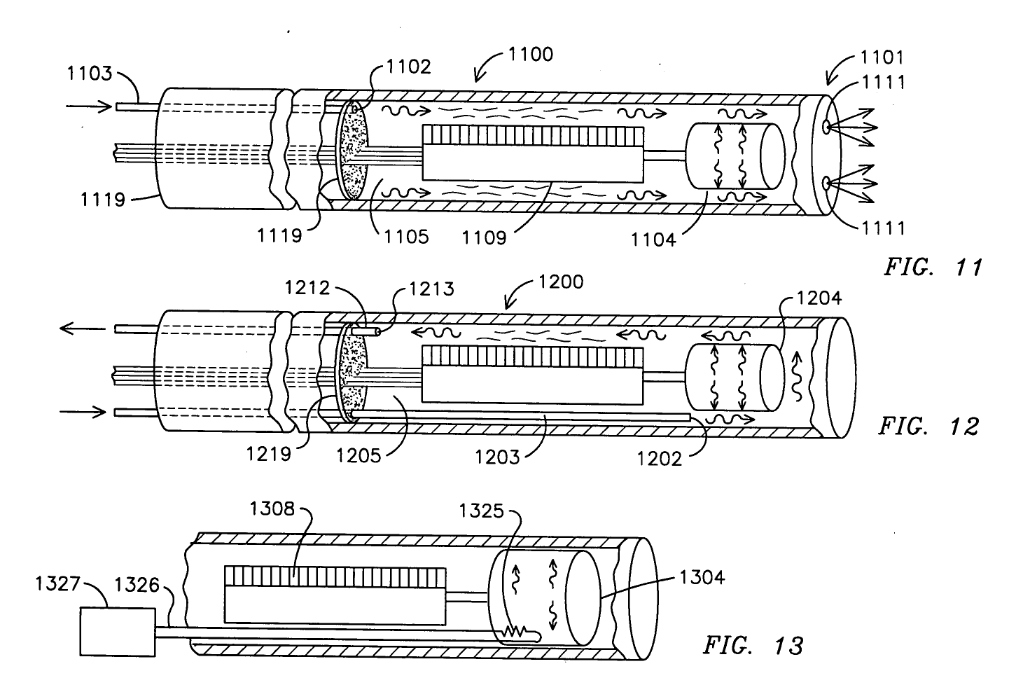

FIG. 12 provides an embodiment of an alternative, closed loop cooling system for a catheter distal end 1200 in which an inlet 1202 opens into a defined space 1205 distal of seal 1219, which may be of any type described herein and in related application Ser. No. ______, filed Jan. 11, 2006, and entitled Apparatus for Catheter Tips, Including Mechanically Scanning Ultrasound Probe Catheter Tip, which is incorporated by reference specifically for such teachings. The inlet 1202 is in fluid communication with a fluid supply source (not shown) via a supply conduit 1203. A return conduit 1212 exiting from catheter distal end 1200 may receive fluid after it passes through the defined space 1205 from the inlet 1202. Any type of outlet may be provided to return fluid, and an outlet 1213 at the end of the return conduit 1212 is not meant to be limiting. As depicted, but also not meant to be limiting, a cool fluid (indicated by arrows) enters the defined space 1205 at inlet 1202 which is near actuator 1204, absorbs heat and exits the defined space 1205 via return conduit 1212. Fluid may be used once or recirculated through a cooling component, such as one external to the catheter.

With regard to the examples of FIGS. 11 and 12, it is further appreciated that open loop and closed loop systems for thermal management may be designed to include fluid flow through a catheter body rather than in distinct conduits as disclosed above. Such embodiments are described in parent application Ser. No. 11/289,926, filed Nov. 30, 2005, for the purpose of providing an acoustic transmission medium, and these teachings are incorporated by reference herein. It is appreciated that such designs and systems may be utilized for thermal management, such as by inclusion of temperature sensors at appropriate locations (e.g., at both ends of the actuator) and an adjustable flow pump to provide a needed flow of fluid to maintain temperature within desired or required limits. Further, and more generally, it is appreciated that aspects of the various embodiments of the apparatuses and systems described herein and in the parent and related application may be designed and used for thermal management of at least one of an heat-generating actuator, a heat-generating transducer, a heat-generating sensor, and a heat-generating therapy component, such as positioned in the defined space within a space-limited catheter, and not for general cooling such as of body tissue that may surround a catheter distal end or tip during use in a body.

More generally regarding temperature sensors, a thermistor or other type of temperature sensor may measure temperature at desired locations or on a particular component. For example, as depicted in FIG. 13, a temperature sensor 1325 is positioned on actuator 1304, and an electrical conduit 1326 passes from sensor 1325 to a control system 1327. If during operation the sensor 1325 exceeds a specified temperature, then the actuator 1304 is shut off. This shut off may be temporary. Alternatively, a program may be initiated that lowers the rate of scanning to reduce temperature generation, and/or a lower power transmission to the transducer 1308. A thermistor or other type of temperature sensor may be provided with any of the above embodiments or combinations thereof, and in any of these may be provided at one or more desired locations, including one or more locations along a catheter or other probe outer wall.

Further to the transducers described above, but not meant to be limiting, an ultrasound transducer may additionally be associated with a backing layer to dampen and thereby shorten pulse duration, and an electrical connection layer. The electrical connection layer may provide electrical communication between electrical conduits passing to the transducer and an interconnect that communicates through a catheter channel to an ultrasound control system, where electrical signals are generated to produce ultrasound signals and where ultrasound data is collected and analyzed. Further, it is appreciated that by ‘transducer’ is meant any known type of transducer which may include a transducer array, such as a 1D, or a 2D array, which may include a phased array. The approaches described above may be provided in various combinations to achieve a desired level of thermal management of probes, including ultrasonic imaging and ultrasound therapy assemblies in catheter distal ends, such as in catheter tips. For a catheter distal end or other probe comprising an actuator to oscillate a transducer in a back and forth motion in order to generate an ultrasound image, such as a 3D imaged volume, non-limiting examples include:
1. The actuator is immersed in a thermally conducting fluid bath (which may also be dielectric and/or a suitable acoustic transmission medium) and also is in close contact with a metal conducting layer of an outer wall (including all variations and additions above, such as exposed metal material between motor mounts).
2. The actuator is immersed in a thermally conducting fluid bath (which may also be dielectric and/or a suitable acoustic transmission medium) and a small propeller, such as attached to a drive shaft of the actuator, circulates the fluid of the fluid bath.
3. The actuator is immersed in a thermally conducting fluid bath (which may also be dielectric and/or a suitable acoustic transmission medium) and one or more fins attached to the transducer assembly and/or a portion of the interconnect near the transducer assembly and in the fluid bath, circulates the fluid of the fluid bath.
4. A closed loop flushable catheter system is implemented in a catheter distal end or other probe in which a metal conducting layer additionally transfers heat from the fluid to surrounding tissue.
5. Actuator not in fluid, but in direct contact with the catheter wall and/or metal in catheter, or coupled to the wall or metal by a thermally conductive solid (e.g. metal, filled polymer, etc.).

It is appreciated that methods comprise providing one or more of the above-described thermal management structures in a catheter distal end or other probe, and operating such probe to maintain thermal output to surrounding tissue within a desired and/or regulated temperature or thermal output by passive and/or active approaches using those structures.

All patents, patent applications, patent publications, and other publications referenced herein are hereby incorporated by reference in this application in order to more fully describe the state of the art to which the present invention pertains, to provide such teachings as are generally known to those skilled in the art.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. A catheter distal end comprising:
an actuator for providing movement for a diagnostic or interventional function;
an catheter outer wall comprising a metal braid or a metal layer; and
a defined space within the catheter outer wall to one or more sides of the actuator;
wherein an association between the actuator to the metal braid or the metal layer provides a thermal conveyance effective to dissipate heat from the actuator.

2. The catheter distal end of claim 1, the association additionally comprising a section of exposed metal braid or metal layer extending from the catheter outer wall proximate the actuator.

3. The catheter distal end of claim 1, wherein a portion of the section of exposed metal braid or metal layer contacts the actuator.

4. The catheter distal end of claim 1, additionally comprising motor mounts connecting the actuator to the catheter outer wall, the association additionally comprising a section of exposed metal braid or metal layer extending between the motor mounts from the catheter outer wall proximate the actuator.

5. The catheter distal end of claim 4, wherein a portion of the section of exposed metal braid or metal layer contacts the actuator.

6. The catheter distal end of claim 1, additionally comprising a thermally conducting fluid in the defined space, effective to additionally provide thermal conveyance from the actuator to the metal braid or the metal layer.

7. The catheter distal end of claim 6, additionally comprising a propeller on a drive shaft connected to the actuator, for circulation of the fluid.

8. The catheter distal end of claim 6, wherein said thermally conducting fluid is a dielectric fluid.

9. The catheter distal end of claim 1, additionally comprising a transducer assembly comprising a fin disposed thereon, wherein the actuator is adapted to move the transducer assembly, and the fin is adapted for circulation of a fluid in the defined space.

10. The catheter distal end of claim 9, additionally comprising a fin disposed on a section of interconnect in the defined space, adapted for circulation of a fluid in the defined space.

11. The catheter distal end of claim 3, additionally comprising a fin disposed on a section of interconnect in the defined space, adapted for circulation of a fluid in the defined space.

12. The catheter distal end of claim 9, wherein the catheter distal end comprises a catheter tip.

13. The catheter distal end of claim 1, wherein the catheter distal end comprises a catheter tip.

14. An catheter distal end for ultrasound imaging or therapy, comprising:
an outer wall comprising a proximal end and a distal end, and within which is positioned a transducer assembly, comprising a transducer, an actuator connected by a drive shaft to the transducer assembly, and an interconnect connecting to the transducer array and extending to or through the proximal end;
a defined space between the outer wall and the transducer assembly, the defined space adapted to contain an acoustic transmission medium; and
the transducer assembly additionally comprising at least one fin extending into the defined space for movement of the acoustic transmission medium.

15. The catheter distal end of claim 14, wherein one of the at least one fin extends from a side of the transducer assembly.

16. The catheter distal end of claim 14, wherein one of the at least one fin extends from the interconnect.

17. An catheter distal end for ultrasonic imaging or therapy, comprising:
an outer wall comprising a proximal end and a distal end, and within which is positioned a transducer assembly, comprising a transducer, an actuator connected by a drive shaft to the transducer assembly, and an interconnect connecting to the transducer array and extending to or through the proximal end; and
a defined space between the outer wall and the transducer assembly, the defined space adapted to contain an acoustic transmission medium.

18. The catheter distal end of claim 17, wherein the outer wall comprises a thermal conducting layer selected from a metal braid and a metal layer.

19. The catheter distal end of claim 18, additionally comprising a section of exposed metal braid or metal layer extending from the catheter outer wall proximate the actuator or the transducer.

20. The catheter distal end of claim 19, wherein a portion of the section of exposed metal braid or metal layer contacts the actuator or the transducer.

21. The catheter distal end of claim 18, additionally comprising motor mounts connecting the actuator to the catheter outer wall, additionally comprising a section of exposed metal braid or metal layer extending between the motor mounts from the catheter outer wall proximate the actuator.

22. The catheter distal end of claim 21, wherein a portion of the section of exposed metal braid or metal layer contacts the actuator.

23. The catheter distal end of claim 18, additionally comprising a thermally conducting fluid in the defined space, effective to additionally provide thermal conveyance from the actuator or the transducer to the metal braid or the metal layer.

24. The catheter distal end of claim 23, additionally comprising a propeller on a drive shaft connected to the actuator, for circulation of the thermally conducting fluid.

25. The catheter distal end of claim 23, wherein said thermally conducting fluid is a dielectric fluid.

26. The catheter distal end of claim 18, additionally comprising a transducer assembly comprising a fin disposed thereon, wherein the actuator is adapted to move the transducer assembly, and the fin is adapted for circulation of a fluid in the defined space.

27. The catheter distal end of claim 26, additionally comprising a fin disposed on a section of interconnect in the defined space, adapted for circulation of a fluid in the defined space.

28. The catheter distal end of claim 20, additionally comprising a fin disposed on a section of interconnect in the defined space, adapted for circulation of a fluid in the defined space.

29. The catheter distal end of claim 26, wherein the ultrasonic catheter distal end comprises a catheter tip.

30. The catheter distal end of claim 17, wherein the catheter distal end comprises a catheter tip.

31. A catheter distal end comprising
an outer wall comprising a proximal end and a distal end, a defined space therein, and
at least one of an heat-generating actuator and a heat-generating transducer positioned in the defined space
wherein the defined space is adapted to receive a thermally conductive fluid and wherein an inlet and an outlet are provided for circulation of said fluid for thermal management of heat generated by the actuator and/or transducer.

32. The catheter distal end of claim 31, wherein a supply conduit for the inlet and a return conduit for the outlet communicate through the proximal end for closed loop thermal management.

33. The catheter distal end of claim 31, wherein a supply conduit for the inlet communicates through the proximal end and the outlet is provided at or near the distal end for open loop thermal management.

34. A catheter system comprising the catheter distal end of claim 31 and a catheter body joined to the catheter distal end, wherein a supply conduit extends through the catheter body, wherein fluid may pass through the supply conduit to the catheter distal end for thermal management.
